SEQUENCE LISTING (12 sequences)

SEQ ID NO: 1 - RNA, 12 nt, Artificial Sequence
  Synthetic construct
gaggaacuncug

SEQ ID NO: 2 - RNA, 11 nt, Artificial Sequence
  Synthetic construct
cguncagccuc

SEQ ID NO: 3 - RNA, 10 nt, Artificial Sequence
  Synthetic construct
guuaguanga

SEQ ID NO: 4 - RNA, 13 nt, Artificial Sequence
  Synthetic construct
gucuagccauggc

SEQ ID NO: 5 - RNA, 13 nt, Artificial Sequence
  Synthetic construct
nggnngacngggu

SEQ ID NO: 6 - RNA, 15 nt, Artificial Sequence
  Synthetic construct
acccncucnaugccn

SEQ ID NO: 7 - RNA, 31 nt, Artificial Sequence
  Synthetic construct
ugcggaaccggugaguacaccggaaungccn

SEQ ID NO: 8 - RNA, 24 nt, Artificial Sequence
  Synthetic construct
ngganauuugggcgugcccccgca

SEQ ID NO: 9 - RNA, 27 nt, Artificial Sequence
  Synthetic construct
gccgaguagnguugggungcgaaaggc

SEQ ID NO: 10 - RNA, 17 nt, Artificial Sequence
  Synthetic construct
gccucccgggagagcca

SEQ ID NO: 11 - RNA, 47 nt, Artificial Sequence
  Synthetic construct
ugguacugccugauagggugcuugcgagugccccgggaggucucgua

SEQ ID NO: 12 - RNA, 24 nt, Artificial Sequence
  Synthetic construct
gaccgugcancaugagcacnnnuc

(19) United States
(12) Patent Application Publication
(10) Pub. No.: US 2003/0059443 A1
(43) Pub. Date: Mar. 27, 2003

(54) Molecular interaction sites of hepatitis C virus RNA and methods of modulating the same

(76) Inventor: David J. Ecker, Encinitas, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/225,501

(22) Filed: Aug. 19, 2002

Related U.S. Application Data
(60) Provisional application No. 60/314,236, filed on Aug. 22, 2001.

(51) Int. Cl. C12Q 1/70; C12Q 1/68; C07H 21/02; C07H 21/04; A61K 39/29

(52) U.S. Cl. 424/225.1; 435/5; 435/6; 536/23.1

(57) ABSTRACT

Polynucleotides comprising molecular interaction sites of hepatitis C virus RNA that have particular secondary structure are provided. Methods of using such polynucleotides to screen, virtually or actually, combinatorial libraries of compounds that bind thereto are also provided. Method of modulating the activity of hepatitis C virus RNA by contacting hepatitis C virus RNA or cells containing the same with a compound identified by such virtual or actual screening are also provided.

### CROSS-REFERENCE TO RELATED APPLICATIONS

This application claims priority to U.S. provisional application Serial No. 60/314,236 filed Aug. 22, 2001, which is incorporated herein by reference in its entirety.

### FIELD OF THE INVENTION

The present invention relates to identification of molecular interaction sites of hepatitis C virus RNA, virtual or actual screening of compounds that bind thereto, and to modulating the activity of hepatitis C virus RNA with such compounds identified in the actual or virtual screening.

### BACKGROUND OF THE INVENTION

The predominant form of hepatitis currently resulting from transfusions is not related to the previously characterized hepatitis A virus or hepatitis B virus and has been referred to as Non-A, Non-B Hepatitis (NANBH). NANBH currently accounts for over 90% of cases of post-transfusion hepatitis. Estimates of the frequency of NANBH in transfusion recipients range from 5%-13% for those receiving volunteer blood, or 25-54% for those receiving blood from commercial sources.

Acute NANBH, while often less severe than acute disease caused by hepatitis A or hepatitis B viruses, occasionally leads to severe or fulminant hepatitis. Of greater concern, progression to chronic hepatitis is much more common after NANBH than after either hepatitis A or hepatitis B infection. Chronic NANBH has been reported in 10%-70% of infected individuals. This form of hepatitis can be transmitted even by asymptomatic patients, and frequently progresses to malignant disease such as cirrhosis and hepatocellular carcinoma. Chronic active hepatitis, with or without cirrhosis, is seen in 44%-90% of post-transfusion hepatitis cases. Of those patients who developed cirrhosis, approximately one-fourth died of liver failure.

Chronic active NANBH is a significant problem to haemophiliacs who are dependent on blood products; 5%-11% of haemophiliacs die of chronic end-stage liver disease. Cases of NANBH other than those traceable to blood or blood products are frequently associated with hospital exposure, accidental needle stick, or tattooing. Transmission through close personal contact also occurs, though this is less common for NANBH than for hepatitis B.

The causative agent of the majority of NANBH has recently been identified and is now referred to as hepatitis C virus (HCV). Houghton et al., EP Publication 318,216; Choo et al., Science, 1989, 244, 359-362. Based on serological studies using recombinant DNA-generated antigens it is now clear that HCV is the causative agent of most cases of post-transfusion NANBH. Clones of cDNA prepared from nucleic acid isolated from concentrated virus particles were originally isolated based on their ability to encode polypeptides which reacted with sera from NANBH patients. These clones hybridized with RNA, but not DNA, isolated from infected liver tissue, indicating the presence of an RNA genome. Hybridization analyses and sequencing of the cDNA clones revealed that RNA present in infected liver and particles was the same polarity as that of the coding strand of the cDNAs; in other words, the virus genome is a positive or plus-strand RNA genome. EP Publication 318,216 discloses partial genomic sequences of HCV-1, and teach recombinant DNA methods of cloning and expressing HCV sequences and HCV polypeptides, techniques of HCV immunodiagnostics, HCV probe diagnostic techniques, anti-HCV antibodies, and methods of isolating new HCV sequences. EP Publication 318,216 also disclose additional HCV sequences and teach application of these sequences and polypeptides in immunodiagnostics, probe diagnostics, anti-HCV antibody production, PCR technology and recombinant DNA technology. Oligomer probes and primers based on the sequences disclosed are also provided. EP Publication 419,182 discloses new HCV isolates J1 and J7 and use of sequences distinct from HCV-1 sequences for screens and diagnostics. Significant improvements in antiviral therapy are therefore greatly desired.

The 5′ untranslated region (5′ UTR) of HCV contains an internal ribosome entry site (IRES) that drives cap-independent initiation of translation of the viral message. Kieft et al., J. Mol. Biol., 1999, 292, 513-529. The stability of the stem-loop involving the initiator AUG has been demonstrated to control the efficiency of internal translation of HCV RNA. Honda et al., RNA, 1996, 2, 955-968. A phylogenetically conserved stem-loop structure at the 5′ border of the IRES of HCV has been shown to be required for cap-independent viral translation. Honda et al., J. Virol., 1999, 73, 1165-1174. In addition, mutational analysis of a conserved tetraloop in the 5′ UTR of HCV has identified a novel RNA element essential for IRES function. Psaridi et al., FEBS Lett., 1999, 453, 49-53. Further, a common structural core exists in the IRES of picomavirus, HCV and pestivirus. Le et al., Virus Genes, 1996, 12, 135-147. Alterations to both the primary and predicted secondary structure of stem-loop IIIc of HCV 1b 5′ UTR leads to mutants that are severely defective in translation that cannot be complemented in trans by the wild-type 5′ UTR sequence. Tang et al., J. Virol., 1999, 73, 2359-2364. Other regions of the IRES have also been identified. Lemon et al., Seminars in Virology, 1997, 8, 274-288. An RNA pseudoknot is an essential structural element of the IRES of HCV. Wang et al., RNA., 1995, 1, 526-537. In addition, genetic analysis of the IRES on HCV has implied involvement of the highly ordered structure and cell type-specific transacting factors. Kamoshita et al., Virol., 1997, 233, 9-18.

Recent advances in genomics, molecular biology, and structural biology have highlighted how RNA molecules participate in or controls many of the events required to express proteins in cells. Rather than function as simple intermediaries, RNA molecules actively regulate their own transcription from DNA, splice and edit mRNA molecules and tRNA molecules, synthesize peptide bonds in the ribosome, catalyze the migration of nascent proteins to the cell membrane, and provide fine control over the rate of translation of messages. RNA molecules can adopt a variety of unique structural motifs that provide the framework required to perform these functions.

“Small” molecule therapeutics, which bind specifically to structured RNA molecules, are organic chemical molecules that are not polymers. “Small” molecule therapeutics include, for example, the most powerful naturally-occurring antibiotics. For example, the aminoglycoside and macrolide antibiotics are “small” molecules that bind to defined regions in ribosomal RNA (rRNA) structures and work, it is believed, by blocking conformational changes in the RNA required for protein synthesis. In addition, changes in the conformation of RNA molecules have been shown to regulate rates of transcription and translation of mRNA molecules. Small molecules are generally less than 10 kDa.

RNA molecules or groups of related RNA molecules are believed by Applicants to have regulatory regions that are used by the cell to control synthesis of proteins. The cell is believed to exercise control over both the timing and the amount of protein that is synthesized by direct, specific interactions with RNA. This notion is inconsistent with the impression obtained by reading the scientific literature on gene regulation, which is highly focused on transcription. The process of RNA maturation, transport, intracellular localization and translation are rich in RNA recognition sites that provide good opportunities for drug binding. Applicants' invention is directed, inter alia, to finding these regions of RNA molecules, in particular the HCV RNA, in the viral genome. Applicants' invention also makes use of combinatorial chemistry to make and/or screen, actually or virtually, a large number of chemical entities for their ability to bind and/or modulate these drug binding sites.

The determination of potential three dimensional structures of nucleic acids and their attendant structural motifs affords insights into areas such as the study of catalysis by RNA, RNA-RNA interactions, RNA-nucleic acid interactions, RNA-protein interactions, and the recognition of small molecules by nucleic acids. Four general approaches to the generation of model three dimensional structures of RNA have been demonstrated in the literature. All of these employ sophisticated molecular modelling and computational algorithms for the simulation of folding and tertiary interactions within target nucleic acids, such as RNA. Westhof and Altman (Proc. Natl. Acad. Sci., 1994, 91, 5133, incorporated herein by reference in its entirety) have described the generation of a three-dimensional working model of M1 RNA, the catalytic RNA subunit of RNase P from E. coli via an interactive computer modelling protocol. Leveraging the significant body of work in the area of cryo-electron microscopy (cryo-EM) and biochemical studies on ribosomal RNAs, Mueller and Brimacombe (J. Mol. Biol., 1997, 271, 524) have constructed a three dimensional model of E. coli 16S Ribosomal RNA. A method to model nucleic acid hairpin motifs has been developed based on a set of reduced coordinates for describing nucleic acid structures and a sampling algorithm that equilibriates structures using Monte Carlo (MC) simulations (Tung, Biophysical J., 1997, 72, 876, incorporated herein by reference in its entirety). MC-SYM is yet another approach to predicting the three dimensional structure of RNAs using a constraint-satisfaction method. Major et al., Proc. Natl. Acad. Sci., 1993, 90, 9408. The MC-SYM program is an algorithm based on constraint satisfaction that searches conformational space for all models that satisfy query input constraints, and is described in, for example, Cedergren et al., RNA Structure And Function, 1998, Cold Spring Harbor Lab. Press, p.37-75. Three dimensional structures of RNA are produced by that method by the stepwise addition of nucleotide having one or several different conformations to a growing oligonucleotide model.

Westhof and Altman (Proc. Natl. Acad. Sci., 1994, 91, 5133) have described the generation of a three-dimensional working model of M1 RNA, the catalytic RNA subunit of RNase P from E. coli via an interactive computer modelling protocol. This modelling protocol incorporated data from chemical and enzymatic protection experiments, phylogenetic analysis, studies of the activities of mutants and the kinetics of reactions catalyzed by the binding of substrate to M1 RNA. Modelling was performed for the most part as described in the literature. Westhof et al., in “Theoretical Biochemistry and Molecular Biophysics,” Beveridge and Lavery (Eds.), Adenine, N.Y., 1990, 399. In general, starting with the primary sequence of M1 RNA, the stem-loop structures and other elements of secondary structure were created. Subsequent assembly of these elements into a three dimensional structure using a computer graphics station and FRODO (Jones, J. Appl. Crystallogr., 1978, 11, 268) followed by refinement using NUCLIN-NUCLSQ afforded a RNA model that had correct geometries, the absence of bad contacts, and appropriate stereochemistry. The model so generated was found to be consistent with a large body of empirical data on M1 RNA and opens the door for hypotheses about the mechanism of action of RNase P. The models generated by this method, however, are less well resolved that the structures determined via X-ray crystallography.

Mueller and Brimacombe (J. Mol. Biol., 1997, 271, 524, which is incorporated herein by reference in its entirety) have constructed a three dimensional model of E. coli 16S ribosomal RNA using a modelling program called ERNA-3D. This program generates three dimensional structures such as A-form RNA helices and single-strand regions via the dynamic docking of single strands to fit electron density obtained from low resolution diffraction data. After helical elements have been defined and positioned in the model, the configurations of the single strand regions is adjusted, so as to satisfy any known biochemical constraints such as RNA-protein cross-linking and foot-printing data.

A method to model nucleic acid hairpin motifs has been developed based on a set of reduced coordinates for describing nucleic acid structures and a sampling algorithm that equilibrates structures using Monte Carlo (MC) simulations. Tung, Biophysical J., 1997, 72, 876, incorporated herein by reference in its entirety. The stem region of a nucleic acid can be adequately modelled by using a canonical duplex formation. Using a set of reduced coordinates, an algorithm that is capable of generating structures of single stranded loops with a pair of fixed ends was created. This allows efficient structural sampling of the loop in conformational space. Combining this algorithm with a modified Metropolis Monte Carlo algorithm afforded a structure simulation package that simplifies the study of nucleic acid hairpin structures by computational means. Once the RNA subdomains have been identified, they can, if desired, be stabilized by the methods disclosed in U.S. Pat. No. 5,712,096.

While X-ray crystallography is a very powerful technique that can allow for the determination of some secondary and tertiary structure of biopolymeric targets (Erikson et al., Ann. Rep. in Med. Chem., 1992, 27, 271-289), this technique can be an expensive procedure and very difficult to accomplish. Crystallization of biopolymers is extremely challenging, difficult to perform at adequate resolution, and is often considered to be as much an art as a science. Further confounding the utility of X-ray crystal structures in the drug discovery process is the inability of crystallography to reveal insights into the solution-phase, and therefore the biologically relevant, structures of the targets of interest. Some analysis of the nature and strength of interaction between a ligand (agonist, antagonist, or inhibitor) and its target can be performed by ELISA (Kemeny and Challacombe, in ELISA and other Solid Phase Immunoassays: 1988), radioligand binding assays (Berson et al., Clin. 1968; Chard, in “An Introduction to Radioimmunoassay and Related Techniques,” 1982), surface-plasmon resonance (Karlsson et al., 1991, Jonsson et al., Biotechniques, 1991), or scintillation proximity assays (Udenfriend et al., Anal. Biochem., 1987), all cited previously. The radioligand binding assays are typically useful only when assessing the competitive binding of the unknown at the binding site for that of the radioligand and also require the use of radioactivity. The surface-plasmon resonance technique is more straightforward to use, but is also quite costly. Conventional biochemical assays of binding kinetics, and dissociation and association constants are also helpful in elucidating the nature of the target-ligand interactions.

Accordingly, one aspect of the invention identifies molecular interaction sites in hepatitis C virus RNA. These molecular interaction sites, which comprise secondary structural elements, are highly likely to give rise to significant therapeutic, regulatory, or other interactions with “small” molecules and the like. Another aspect of the invention is to compare molecular interaction sites of hepatitis C virus RNA with compounds proposed for interaction therewith.

Yet another aspect of the present invention is the establishment of databases of the numerical representations of three-dimensional structures of molecular interaction sites of hepatitis C virus RNA. Such databases libraries provide powerful tools for the elucidation of structure and interactions of molecular interaction sites with potential ligands and predictions thereof. Another aspect of the present invention is to provide a general method for the screening of combinatorial libraries comprising individual compounds or mixtures of compounds against hepatitis C virus RNA, so as to determine which components of the library bind to the target.

### SUMMARY OF THE INVENTION

The present invention is directed to identification of molecular interaction sites of hepatitis C virus RNA that comprise particular secondary structure.

The present invention is also directed to nucleic acid molecules, polynucleotides or oligonucleotides comprising the molecular interaction sites that can be used to screen, virtually or actually, combinatorial libraries of compounds that bind thereto.

The present invention is also directed to computer-readable medium comprising. three dimensional representations of the structures of the molecular interaction sites.

The present invention is also directed to modulating the activity of hepatitis C virus RNA by contacting hepatitis C virus RNA or prokaryotic cells comprising the same with a compound identified by such virtual or actual screening.

The present invention is also directed to modulating prokaryotic cell growth comprising contacting a prokaryotic cell with a compound identified by such virtual or actual screening.

### BRIEF DESCRIPTION OF THE DRAWINGS

FIGS. 1, 1A, 1B, 1C, 1D, 1E, 1F, 1G and 1H depict representative secondary structures of the 5′ untranslated region (5′ UTR) of hepatitis C virus showing sites 1-8 (nucleotides: capitalized letters=>95% conservation; small letters=90 to 95% conservation; ●−80 to 90% conservation; and ∘=<80% conservation).

### DESCRIPTION OF PREFERRED EMBODIMENTS OF THE INVENTION

The present invention is directed to, inter alia, identification of molecular interaction sites of hepatitis C virus RNA. Such molecular interaction sites comprise secondary structure capable of interacting with cellular components, such as factors and proteins required for translation and other cellular processes. Nucleic acid molecules or polynucleotides comprising the molecular interaction sites can be used to screen, virtually or actually, combinatorial libraries of compounds that bind thereto. The compounds identified by such screening are used to modulate the activity of hepatitis C virus RNA and, thus, can be used to modulate, either inhibit or stimulate, viral replication. Thus, novel drugs, agricultural chemicals, industrial chemicals and the like that operate through the modulation of hepatitis C virus RNA can be identified.

A number of procedures and protocols are preferably integrated to provide powerful drug and other biologically useful compound identification. Pharmaceuticals, veterinary drugs, agricultural chemicals, pesticides, herbicides, fungicides, industrial chemicals, research chemicals and many other beneficial compounds useful in pollution control, industrial biochemistry, and biocatalytic systems can be identified in accordance with embodiments of this invention. Novel combinations of procedures provide extraordinary power and versatility to the present methods. While it is preferred in some embodiments to integrate a number of processes developed by the assignee of the present application as will be set forth more fully herein, it should be recognized that other methodologies can be integrated herewith to good effect. Thus, while it is greatly advantageous to determine molecular binding sited on hepatitis C virus RNA in accordance with the teachings of this invention, the interactions of ligands and libraries of ligands with other hepatitis C virus RNA identified as being of interest may greatly benefit from other aspects of this invention. All such combinations are within the spirit of the invention.

One aspect of Applicants' invention is directed to identifying secondary structures in hepatitis C virus RNA termed “molecular interaction sites.” As used herein, “molecular interaction sites” are regions of hepatitis C virus RNA that have secondary structure. Molecular interaction sites can be conserved among a plurality of different taxonomic species of hepatitis C virus RNA. Molecular interaction sites are small, preferably less than 200 nucleotides, preferably less than 150 nucleotides, preferably less than 70 nucleotides, preferably less than 50 nucleotides, alternatively less than 30 nucleotides, independently folded, functional subdomains contained within a larger RNA molecule. Molecular interaction sites can contain both single-stranded and double-stranded regions. Thus, molecular interaction sites are capable of undergoing interaction with “small” molecules and otherwise, and are expected to serve as sites for interacting with “small” molecules, oligomers such as oligonucleotides, and other compounds in therapeutic and other applications. Molecular interaction sites also comprise a pocket for binding small molecules, drugs and the like.

The molecular interaction sites are present within at least hepatitis C virus RNA. In accordance with some embodiments of this invention, it will be appreciated that the hepatitis C virus RNAs having a molecular interaction site or sites may be derived from a number of sources. Thus, such hepatitis C virus RNAs can be identified by any means, rendered into three dimensional representations and employed for the identification of compounds that can interact with them to effect modulation of the hepatitis C virus RNA. In some embodiments, the molecular interaction sites that are identified in hepatitis C virus RNA are absent from eukaryotes, particularly humans, and, thus, can serve as sites for “small” molecule binding with concomitant modulation of the hepatitis C virus RNA of prokaryotic organisms without effecting human toxicity.

The molecular interaction sites can be identified by any means known to the skilled artisan. In some embodiments of the invention, the molecular interaction sites in hepatitis C virus RNA are identified according to the general methods described in International Publication WO 99/58719, which is incorporated herein by reference in its entirety. Briefly, a target hepatitis C virus RNA nucleotide sequence is chosen from among known sequences. Any hepatitis C virus RNA nucleotide sequence can be chosen. The nucleotide sequence of the target hepatitis C virus RNA is compared to the nucleotide sequences of a plurality of hepatitis C virus RNAs from different isolates. At least one sequence region that is effectively conserved among the plurality of hepatitis C virus RNAs and the target hepatitis C virus RNA is identified. Such conserved region is examined to determine whether there is any secondary structure, and, for conserved regions having secondary structure, such secondary structure is identified.

In accordance with some embodiments of the invention, the nucleotide sequence of the target hepatitis C virus RNA is compared with the nucleotide sequences of a plurality of corresponding hepatitis C virus RNAs from different isolates. Initial selection of a particular target nucleic acid can be based upon any functional criteria. Additional hepatitis C virus RNA targets can be determined independently or can be selected from publicly available genetic databases known to those skilled in the art. Databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet at, for example, ncbi.nlm.nih.gov/Omim/. CGAP, which is an interdisciplinary program to establish the information and technological tools required to decipher the molecular anatomy of a cancer cell, can be accessed through the world wide web of the Internet at, for example, ncbi.nlm.nih.gov/ncicgap/. Some of these databases may contain complete or partial nucleotide sequences. In addition, hepatitis C virus RNA targets can also be selected from private genetic databases. Alternatively, hepatitis C virus RNA targets can be selected from available publications or can be determined especially for use in connection with the present invention.

After a hepatitis C virus RNA target is selected or provided, the nucleotide sequence of the hepatitis C virus RNA target is determined and then compared to the nucleotide sequences of a plurality of hepatitis C virus RNAs from different isolates. In one embodiment of the invention, the nucleotide sequence of the hepatitis C virus RNA target is determined by scanning at least one genetic database or is identified in available publications. Databases known and available to those skilled in the art include, for example, GenBank, and the like. These databases can be used in connection with searching programs such as, for example, Entrez, which is known and available to those skilled in the art, and the like. Entrez can be accessed through the world wide web of the Internet at, for example, ncbi.nlm.nih.gov/Entrez/. Preferably, the most complete nucleic acid sequence representation available from various databases is used. The GenBank database, which is known and available to those skilled in the art, can also be used to obtain the most complete nucleotide sequence. GenBank is the NIH genetic sequence database and is an annotated collection of all publicly available DNA sequences. GenBank is described in, for example, Nuc. Acids Res., 1998, 26, 1-7, which is incorporated herein by reference in its entirety, and can be accessed by those skilled in the art through the world wide web of the Internet at, for example, ncbi.nlm.nih.gov/Web/Genbank/index.html. Alternatively, partial nucleotide sequences of hepatitis C virus RNA targets can be used when a complete nucleotide sequence is not available.

The nucleotide sequence of the hepatitis C virus RNA target is compared to the nucleotide sequences of a plurality of hepatitis C virus RNAs from different isolates. A plurality of hepatitis C virus RNAs from different isolates, and the nucleotide sequences thereof, can be found in genetic databases, from available publications, or can be determined especially for use in connection with the present invention. In one embodiment of the invention, the hepatitis C virus RNA target is compared to the nucleotide sequences of a plurality of hepatitis C virus RNAs from different isolates by performing a sequence similarity search, an ortholog search, or both, such searches being known to persons of ordinary skill in the art.

The result of a sequence similarity search is a plurality of hepatitis C virus RNAs having at least a portion of their nucleotide sequences which are homologous to at least an 8 to 20 nucleotide region of the target hepatitis C virus RNA, referred to as the window region. Preferably, the plurality of hepatitis C virus RNAs comprise at least one portion which is at least 60% homologous to any window region of the target hepatitis C virus RNA. More preferably, the homology is at least 70%. More preferably, the homology is at least 80%. Most preferably, the homology is at least 90% or 95%. For example, the window size, the portion of the target hepatitis C virus RNA to which the plurality of sequences are compared, can be from about 8 to about 20, preferably from about 10 to about 15, most preferably from about 11 to about 12, contiguous nucleotides. The window size can be adjusted accordingly. A plurality of hepatitis C virus RNAs from different isolates is then preferably compared to each likely window in the target hepatitis C virus RNA until all portions of the plurality of sequences is compared to the windows of the target hepatitis C virus RNA. Sequences of the plurality of hepatitis C virus RNAs from different isolates which have portions which are at least 60%, preferably at least 70%, more preferably at least 80%, or most preferably at least 90% homologous to any window sequence of the target hepatitis C virus RNA are considered as likely homologous sequences.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet at, for example, ncbi.nlm.nih.gov/BLAST/. The GCG Package provides a local version of Blast that can be used either with public domain databases or with any locally available searchable database. GCG Package v.9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the world wide web of the Internet at, for example, gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

Another toolkit capable of doing sequence similarity searching and data manipulation is SEALS, also from NCBI. This tool set is written in perl and C and can run on any computer platform that supports these languages. It is available for download, for example, at the world wide web of the Internet at ncbi.nlm.nih.gov/Walker/SEALS/. This toolkit provides access to Blast2 or gapped blast. It also includes a tool called tax_collector which, in conjunction with a tool called tax_break, parses the output of Blast2 and returns the identifier of the sequence most homologous to the query sequence for each isolate present. Another useful tool is feature2fasta which extracts sequence fragments from an input sequence based on the annotation.

Preferably, the plurality of hepatitis C virus RNAs from different isolates that have homology to the target nucleic acid, as described above in the sequence similarity search, are further delineated so as to find orthologs of the target hepatitis C virus RNA therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing an ortholog search, an exhaustive list of homologous sequences from different isolates is obtained. Subsequently, these sequences are analyzed to select the best representative sequence that fits the criteria for being an ortholog. An ortholog search can be performed by programs available to those skilled in the art including, for example, Compare. Preferably, an ortholog search is performed with access to complete and parsed GenBank annotations for each of the sequences. Currently, the records obtained from GenBank are “flat-files”, and are not ideally suited for automated analysis. Preferably, the ortholog search is performed using a Q-Compare program. The Blast Results-Relation database and the Annotations-Relational database are used in the Q-Compare protocol, which results in a list of ortholog sequences to compare in the interspecies sequence comparisons programs described below.

The above-described similarity searches provide results based on cut-off values, referred to as e-scores. E-scores represent the probability of a random sequence match within a given window of nucleotides. The lower the e-score, the better the match. One skilled in the art is familiar with e-scores. The user defines the e-value cut-off depending upon the stringency, or degree of homology desired, as described above. In some embodiments of the invention, it is preferred that any homologous nucleotide sequences of hepatitis C virus RNA that are identified not be present in the human genome.

In another embodiment of the invention, the sequences required are obtained by searching ortholog databases. One such database is Hovergen, which is a curated database of vertebrate orthologs. Ortholog sets may be exported from this database and used as is, or used as seeds for further sequence similarity searches as described above. Further searches may be desired, for example, to find invertebrate orthologs. Hovergen can be downloaded as a file transfer program at, for example, pbil.univ-lyon1.fr/pub/hovergen/. A database of prokaryotic orthologs, COGS, is available and can be used interactively through the world wide web of the Internet at, for example, ncbi.nlm.nih.gov/COG/.

After the orthologs or virtual transcripts described above are obtained through either the sequence similarity search or the ortholog search, at least one sequence region which is conserved among the plurality of hepatitis C virus RNAs from different isolates and the target hepatitis C virus RNA is identified. Sequence comparisons can be performed using numerous computer programs which are available and known to those skilled in the art. Preferably, interspecies sequence comparison is performed using Compare, which is available and known to those skilled in the art. Compare is a GCG tool that allows pair-wise comparisons of sequences using a window/stringency criterion. Compare produces an output file containing points where matches of specified quality are found. These can be plotted with another GCG tool, DotPlot.

Alternatively, the identification of a conserved sequence region is performed by interspecies sequence comparisons using the ortholog sequences generated from Q-Compare in combination with CompareOverWins. Preferably, the list of sequences to compare, i.e., the ortholog sequences, generated from Q-Compare is entered into the CompareOverWins algorithm. Preferably, interspecies sequence comparisons are performed by a pair-wise sequence comparison in which a query sequence is slid over a window on the master target sequence. Preferably, the window is from about 9 to about 99 contiguous nucleotides.

Sequence homology between the window sequence of the target hepatitis C virus RNA and the query sequence of any of the plurality of hepatitis C virus RNAs obtained as described above, is preferably at least 60%, more preferably at least 70%, more preferably at least 80%, and most preferably at least 90% or 95%. The most preferable method of choosing the threshold is to have the computer automatically try all thresholds from 50% to 100% and choose a threshold based a metric provided by the user. One such metric is to pick the threshold such that exactly n hits are returned, where n is usually set to 3. This process is repeated until every base on the query nucleic acid, which is a member of the plurality of hepatitis C virus RNAs described above, has been compared to every base on the master target sequence. The resulting scoring matrix can be plotted as a scatter plot. Based on the match density at a given location, there may be no dots, isolated dots, or a set of dots so close together that they appear as a line. The presence of lines, however small, indicates primary sequence homology. Sequence conservation within hepatitis C virus RNA in divergent isolates is likely to be an indicator of conserved regulatory elements that are also likely to have a secondary structure. The results of the interspecies sequence comparison can be analyzed using MS Excel and visual basic tools in an entirely automated manner as known to those skilled in the art.

After at least one region that is conserved between the nucleotide sequence of the hepatitis C virus RNA target and the plurality of hepatitis C virus RNA s from different isolates, preferably via the orthologs, is identified, the conserved region is analyzed to determine whether it contains secondary structure. Determining whether the identified conserved regions contain secondary structure can be performed by a number of procedures known to those skilled in the art. Determination of secondary structure is preferably performed by self complementarity comparison, alignment and covariance analysis, secondary structure prediction, or a combination thereof.

In one embodiment of the invention, secondary structure analysis is performed by alignment and covariance analysis. Numerous protocols for alignment and covariance analysis are known to those skilled in the art. Preferably, alignment is performed by ClustalW, which is available and known to those skilled in the art. ClustalW is a tool for multiple sequence alignment that, although not a part of GCG, can be added as an extension of the existing GCG tool set and used with local sequences. ClustalW can be accessed through the world wide web of the Internet at, for example, dot.imgen.bcm.tmc.edu:9331/multi-align/Options/clustalw.html. ClustalW is also described in Thompson, et al., Nuc. Acids Res., 1994, 22, 4673-4680, which is incorporated herein by reference in its entirety. These processes can be scripted to automatically use conserved UTR regions identified in earlier steps. Seqed, a UNIX command line interface available and known to those skilled in the art, allows extraction of selected local regions from a larger sequence. Multiple sequences from many different isolates can be clustered and aligned for further analysis.

In another embodiment of the invention, the output of all possible pair-wise CompareOverWindows comparisons are compiled and aligned to a reference sequence using a program called AlignHits, a program that can be reproduced by one skilled in the art. One purpose of this program is to map all hits made in pair-wise comparisons back to the position on a reference sequence. This method combining CompareOverWindows and AlignHits provides more local alignments (over 20-100 bases) than any other algorithm. This local alignment is required for the structure finding routines described later such as covariation or RevComp. This algorithm writes a fasta file of aligned sequences. It is important to differentiate this from using ClustalW by itself, without CompareOverWindows and AlignHits.

Covariation is a process of using phylogenetic analysis of primary sequence information for consensus secondary structure prediction. Covariation is described in the following references, each of which is incorporated herein by reference in their entirety: Gutell et al., “Comparative Sequence Analysis Of Experiments Performed During Evolution” In Ribosomal RNA Group I Introns, Green, Ed., Austin: Landes, 1996; Gautheret et al., Nuc. Acids Res., 1997, 25, 1559-1564; Gautheret et al., RNA, 1995, 1, 807-814; Lodmell et al., Proc. Natl. Acad. Sci. USA, 1995, 92, 10555-10559; Gautheret et al., J. Mol. Biol., 1995, 248, 27-43; Gutell, Nuc. Acids Res., 1994, 22, 3502-3517; Gutell, Nuc. Acids Res., 1993, 21, 3055-3074; Gutell, Nuc. Acids Res., 1993, 21, 3051-3054; Woese, Proc. Natl. Acad. Sci. USA, 1989, 86, 3119-3122; and Woese et al., Nuc. Acids Res., 1980, 8, 2275-2293, each of which is incorporated herein by reference in its entirety. Preferably, covariance software is used for covariance analysis. Preferably, Covariation, a set of programs for the comparative analysis of RNA structure from sequence alignments, is used. Covariation uses phylogenetic analysis of primary sequence information for consensus secondary structure prediction. Covariation can be obtained through the world wide web of the Internet at, for example, mbio.ncsu.edu/RNaseP/info/programs/programs.html. A complete description of a version of the program has been published (Brown, J. W. 1991, Phylogenetic analysis of RNA structure on the Macintosh computer. CABIOS 7:391-393). The current version is v4.1, which can perform various types of covariation analysis from RNA sequence alignments, including standard covariation analysis, the identification of compensatory base-changes, and mutual information analysis. The program is well-documented and comes with extensive example files. It is compiled as a stand-alone program; it does not require Hypercard (although a much smaller ‘stack’ version is included). This program will run in any Macintosh environment running MacOS v7.1 or higher. Faster processor machines (68040 or PowerPC) is suggested for mutual information analysis or the analysis of large sequence alignments.

In another embodiment of the invention, secondary structure analysis is performed by secondary structure prediction. There are a number of algorithms that predict RNA secondary structures based on thermodynamic parameters and energy calculations. Preferably, secondary structure prediction is performed using either M-fold or RNA Structure 2.52. M-fold can be accessed through the world wide web of the Internet at, for example, ibc.wustl.edu/-zuker/ma/form2.cgi or can be downloaded for local use on UNIX platforms. M-fold is also available as a part of GCG package. RNA Structure 2.52 is a windows adaptation of the M-fold algorithm and can be accessed through the world wide web of the Internet at, for example, 128.151.176.70/RNAstructure.html.

In another embodiment of the invention, secondary structure analysis is performed by self complementarity comparison. Preferably, self complementarity comparison is performed using Compare, described above. More preferably, Compare can be modified to expand the pairing matrix to account for G-U or U-G basepairs in addition to the conventional Watson-Crick G-C/C-G or A-U/U-A pairs. Such a modified Compare program (modified Compare) begins by predicting all possible base-pairings within a given sequence. As described above, a small but conserved region is identified based on primary sequence comparison of a series of orthologs. In modified Compare, each of these sequences is compared to its own reverse complement. Allowable base-pairings include Watson-Crick A-U, G-C pairing and non-canonical G-U pairing. An overlay of such self complementarity plots of all available orthologs, and selection for the most repetitive pattern in each, results in a minimal number of possible folded configurations. These overlays can then used in conjunction with additional constraints, including those imposed by energy considerations described above, to deduce the most likely secondary structure.

In another embodiment of the invention, the output of AlignHits is read by a program called RevComp. This program could be reproduced by one skilled in the art. One purpose of this program is to use base pairing rules and ortholog evolution to predict RNA secondary structure. RNA secondary structures are composed of single stranded regions and base paired regions, called stems. Since structure conserved by evolution is searched, the most probable stem for a given alignment of ortholog sequences is the one which could be formed by the most sequences. Possible stem formation or base pairing rules is determined by, for example, analyzing base pairing statistics of stems which have been determined by other techniques such as NMR. The output of RevComp is a sorted list of possible structures, ranked by the percentage of ortholog set member sequences which could form this structure. Because this approach uses a percentage threshold approach, it is insensitive to noise sequences. Noise sequences are those that either not true orthologs, or sequences that made it into the output of AlignHits due to high sequence homology even though they do not represent an example of the structure which is searched. A very similar algorithm is implemented using Visual basic for Applications (VBA) and Microsoft Excel to be run on PCs, to generate the reverse complement matrix view for the given set of sequences.

A result of the secondary structure analysis described above, whether performed by alignment and covariance, self complementarity analysis, secondary structure predictions, such as using M-fold or otherwise, is the identification of secondary structure in the conserved regions among the target hepatitis C virus RNA and the plurality of hepatitis C virus RNAs from different isolates. Exemplary secondary structures that may be identified include, but are not limited to, bulges, loops, stems, hairpins, knots, triple interacts, cloverleafs, or helices, or a combination thereof. Alternatively, new secondary structures may be identified.

The present invention is also directed to nucleic acid molecules, such as polynucleotides and oligonucleotides, comprising a molecular interaction site present in hepatitis C virus RNA. Nucleic acid molecules include the physical compounds themselves as well as in silico representations of the same. Thus, the nucleic acid molecules are derived from hepatitis C virus RNA. The molecular interaction site serves as a binding site for at least one molecule which, when bound to the molecular interaction site, modulates the expression of the hepatitis C virus RNA in a cell. The nucleotide sequence of the polynucleotide is selected to provide the secondary structure of the molecular interaction sites described in grater detail in the Examples. The nucleotide sequence of the polynucleotide is preferably the nucleotide sequence of the target hepatitis C virus RNAs, described above. Alternatively, the nucleotide sequence is preferably the nucleotide sequence of hepatitis C virus RNAs from a plurality of different isolates which also contain the molecular interaction site.

The polynucleotides of the invention comprise the molecular interaction sites of the hepatitis C virus RNA. Thus, the polynucleotides of the invention comprise the nucleotide sequences of the molecular interaction sites. In addition, the polynucleotides can comprise up to 50, more preferably up to 40, more preferably up to 30, more preferably up to 20, and most preferably up to 10 additional nucleotides at either the 5′ or 3′, or combination thereof, ends of each polynucleotide. Thus, for example, if a molecular interaction site comprises 25 nucleotides, the polynucleotide can comprise up to 75 nucleotides. The nucleotides that are in addition to those present in the molecular interaction site are selected to preserve the secondary structure of the molecular interaction site. One skilled in the art can select such additional nucleotides so as to conserve the secondary structure. The polynucleotides can comprise either RNA or DNA or can be chimeric RNA/DNA. The polynucleotides can comprise modified bases, sugars and backbones that are well known to the skilled artisan. Further, a single polynucleotide can comprise a plurality of molecular interaction sites. In addition, a plurality of polynucleotides can, together, comprise a single molecular interaction site. Alternatively, when a plurality of polynucleotides together comprise a molecular interaction site, one skilled in the art can attach the polynucleotides to one another, thus, forming a single polynucleotide.

The portion of the polynucleotide comprising the molecular interaction site can comprise one or more deletions, insertions and substitutions. Stems, end loops, bulges, internal loops, and dangling regions can comprise one or more deletions, insertions and substitutions. Thus, for example, an end loop of a molecular interaction site that consists of 10 nucleotides can be modified to contain one or more insertions, deletions or substitutions, thus, resulting in a shortening or lengthening of the stem preceding the end loop. In addition, unpaired, dangling nucleotides that are adjacent to, for example, a double-stranded region can be deleted or can be basepaired with the addition of another nucleotide, thus, lengthening the stem. In addition, nucleotide base pairings within a stem can also be substituted, deleted, or inserted. Thus, for example, an A-U basepair within a stem portion of a molecular interaction site can be replaced with a G-C basepair. Further, non-canonical base pairing (e.g., G-A, C-T, G-U, etc.) can also be present within the polynucleotide. Thus, polynucleotides having at least 70%, more preferably 80%, more preferably 90%, more preferably 95%, and most preferably 99% homology with the molecular interaction sites, such as those set forth in the Examples below, are included within the scope of the invention. Percent homology can be determined by, for example, the Gap program (Wisconsin Sequence Analysis Package, Version 8 for Unix, Genetics Computer Group, University Research Park, Madison Wis.), using the default settings, which uses the algorithm of Smith and Waterman (Adv. Appl. Math., 1981, 2, 482-489, which is incorporated herein by reference in its entirety).

The present invention is also directed to the purified and isolated nucleic acid molecules, or polynucleotides, described above, that are present within hepatitis C virus RNA. The polynucleotides comprising the molecular interaction site mimic the portion of the hepatitis C virus RNA comprising the molecular interaction site.

Polynucleotides, and modifications thereof, are well known to those skilled in the art. The polynucleotides of the invention can be used, for example, as research reagents to detect, for example, naturally occurring molecules that bind the molecular interaction sites. Alternatively, the polynucleotides of the invention can be used to screen, either actually or virtually, small molecules that bind the molecular interaction sites, as described below in greater detail. Virtual generation of compounds and screening thereof for binding to molecular interaction sites is described in, for example, International Publication WO 99/58947, which is incorporated herein by reference in its entirety. The polynucleotides of the invention can also be used as decoys to compete with naturally-occurring molecular interaction sites within a cell for research, diagnostic and therapeutic applications. In particular, the polynucleotides can be used in, for example, therapeutic applications to inhibit bacterial growth. Molecules that bind to the molecular interaction site modulate, either by augmenting or diminishing, the function of hepatitis C virus RNA in translation. The polynucleotides can also be used in agricultural, industrial and other applications.

The present invention is also directed to compositions comprising at least one polynucleotide described above. In some embodiments of the invention, two polynucleotides are included within a composition. The compositions of the invention can optionally comprise a carrier. A “carrier” is an acceptable solvent, diluent, suspending agent or any other inert vehicle for delivering one or more nucleic acids to an animal, and are well known to those skilled in the art. The carrier can be a pharmaceutically acceptable carrier. The carrier can be liquid or solid and is selected, with the planned manner of administration in mind, so as to provide for the desired bulk, consistency, etc., when combined with the other components of the composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.).

The present invention is also directed to methods of identifying compounds that bind to a molecular interaction site of hepatitis C virus RNA comprising providing a numerical representation of the three-dimensional structure of the molecular interaction site and providing a compound data set comprising numerical representations of the three dimensional structures of a plurality of organic compounds. The numerical representation of the molecular interaction site is then compared with members of the compound data set to generate a hierarchy of organic compounds ranked in accordance with the ability of the organic compounds to form physical interactions with the molecular interaction site.

The present invention is also directed to methods of identifying compounds that bind to a molecular interaction site of hepatitis C virus RNA, or a polynucleotide comprising the same. In some embodiments of the invention, compounds that bind to a molecular interaction site of hepatitis C virus RNA, or a polynucleotide comprising the same, are identified according to the general methods described in International Publication WO 99/58947, which is incorporated herein by reference in its entirety. Briefly, the methods comprise providing a numerical representation of the three dimensional structure of the molecular interaction site, or a polynucleotide comprising the same, providing a compound data set comprising numerical representations of the three dimensional structures of a plurality of organic compounds, comparing the numerical representation of the molecular interaction site with members of the compound data set to generate a hierarchy of organic compounds which is ranked in accordance with the ability of the organic compounds to form physical interactions with the molecular interaction site.

While there are a number of ways to characterize binding between molecular interaction sites and ligands, such as for example, organic compounds, methodologies are described in International Publications WO 99/58719, WO 99/59061, WO 99/58722, WO 99/45150, WO 99/58474, and WO 99/58947, each of which is assigned to the assignee of the present inventions, and each of which is incorporated by reference herein in their entirety.

In addition, the present invention is also directed to three dimensional representations of the nucleic acid molecules, and compositions comprising the same, described above. The three dimensional structure of a molecular interaction site of hepatitis C virus RNA can be manipulated as a numerical representation. The three dimensional representations, i.e., in silico (e.g. in computer-readable form) representations can be generated by methods disclosed in, for example, International Publication WO 99/58947, which is incorporated herein by reference in its entirety. Briefly, the three dimensional structure of a molecular interaction site, preferably of an RNA, can be manipulated as a numerical representation. Computer software that provides one skilled in the art with the ability to design molecules based on the chemistry being performed and on available reaction building blocks is commercially available. Software packages such as, for example, Sybyl/Base (Tripos, St. Louis, Mo.), Insight II (Molecular Simulations, San Diego, Calif.), and Sculpt (MDL Information Systems, San Leandro, Calif.) provide means for computational generation of structures. These software products also provide means for evaluating and comparing computationally generated molecules and their structures. In silico collections of molecular interaction sites can be generated using the software from any of the above-mentioned vendors and others which are or may become available. The three dimensional representations can be used, for example, to dock the molecule(s) to potential therapeutic compounds. Thus, the three dimensional representations can be used in drug screening procedures. Accordingly, the nucleic acid molecules and compositions comprising the same of the present invention include the three dimensional representations of the same.

A set of structural constraints for the molecular interaction site of the hepatitis C virus RNA can be generated from biochemical analyses such as, for example, enzymatic mapping and chemical probes, and from genomics information such as, for example, covariance and sequence conservation. Information such as this can be used to pair bases in the stem or other region of a particular secondary structure. Additional structural hypotheses can be generated for noncanonical base pairing schemes in loop and bulge regions. A Monte Carlo search procedure can sample the possible conformations of the hepatitis C virus RNA consistent with the program constraints and produce three dimensional structures.

Reports of the generation of three dimensional, in silico representations are available from the standpoint of library design, generation, and screening against protein targets. Likewise, some efforts in the area of generating RNA models have been reported in the literature. However, there are no reports on the use of structure-based design approaches to query in silico representations of organic molecules, “small” molecules, polynucleotides or other nucleic acids, with three dimensional, in silico, representations of hepatitis C virus RNA structures. The present invention preferably employs computer software that allows the construction of three dimensional models of hepatitis C virus RNA structure, the construction of three dimensional, in silico representations of a plurality of organic compounds, “small” molecules, polymeric compounds, polynucleotides and other nucleic acids, screening of such in silico representations against hepatitis C virus RNA molecular interaction sites in silico, scoring and identifying the best potential binders from the plurality of compounds, and finally, synthesizing such compounds in a combinatorial fashion and testing them experimentally to identify new ligands for such hepatitis C virus RNA targets.

The molecules that may be screened by using the methods of this invention include, but are not limited to, organic or inorganic, small to large molecular weight individual compounds, and combinatorial mixture or libraries of ligands, inhibitors, agonists, antagonists, substrates, and biopolymers, such as peptides or polynucleotides. Combinatorial mixtures include, but are not limited to, collections of compounds, and libraries of compounds. These mixtures may be generated via combinatorial synthesis of mixtures or via admixture of individual compounds. Collections of compounds include, but are not limited to, sets of individual compounds or sets of mixtures or pools of compounds. These combinatorial libraries may be obtained from synthetic or from natural sources such as, for example to, microbial, plant, marine, viral and animal materials. Combinatorial libraries include at least about twenty compounds and as many as a thousands of individual compounds and potentially even more. When combinatorial libraries are mixtures of compounds these mixtures typically contain from 20 to 5000 compounds preferably from 50 to 1000, more preferably from 50 to 100. Combinations of from 100 to 500 are useful as are mixtures having from 500 to 1000 individual species. Typically, members of combinatorial libraries have molecular weight less than about 10,000 Da, more preferably less than 7,500 Da, and most preferably less than 5000 Da.

A significant advance in the area of virtual screening was the development of a software program called DOCK that allows structure-based database searches to find and identify the interactions of known molecules to a receptor of interest (Kuntz et al., Acc. Chem. Res., 1994, 27, 117; Geschwend and Kuntz, J. Compt.-Aided Mol. Des., 1996, 10, 123). DOCK allows the screening of molecules, whose 3D structures have been generated in silico, but for which no prior knowledge of interactions with the receptor is available. DOCK, therefore, provides a tool to assist in discovering new ligands to a receptor of interest. DOCK can thus be used for docking the compounds prepared according to the methods of the present invention to desired target molecules. Implementation of DOCK is described in, for example, International Publication WO 99/58947, which is incorporated herein by reference in its entirety.

In some embodiments of the invention, an automated computational search algorithm, such as those described above, is used to predict all of the allowed three dimensional molecular interaction site structures from hepatitis C virus RNA, which are consistent with the biochemical and genomic constraints specified by the user. Based, for example, on their root-mean-squared deviation values, these structures are clustered into different families. A representative member or members of each family can be subjected to further structural refinement via molecular dynamics with explicit solvent and cations.

Structural enumeration and representation by these software programs is typically done by drawing molecular scaffolds and substituents in two dimensions. Once drawn and stored in the computer, these molecules may be rendered into three dimensional structures using algorithms present within the commercially available software. Preferably, MC-SYM is used to create three dimensional representations of the molecular interaction site. The rendering of two dimensional structures of molecular interaction sites into three dimensional models typically generates a low energy conformation or a collection of low energy conformers of each molecule. The end result of these commercially available programs is the conversion of a hepatitis C virus RNA sequence containing a molecular interaction site into families of similar numerical representations of the three dimensional structures of the molecular interaction site. These numerical representations form an ensemble data set.

The three dimensional structures of a plurality of compounds, preferably “small” organic compounds, can be designated as a compound data set comprising numerical representations of the three dimensional structures of the compounds. “Small” molecules in this context refers to non-oligomeric organic compounds. Two dimensional structures of compounds can be converted to three dimensional structures, as described above for the molecular interaction sites, and used for querying against three dimensional structures of the molecular interaction sites. The two dimensional structures of compounds can be generated rapidly using structure rendering algorithms commercially available. The three dimensional representation of the compounds which are polymeric in nature, such as polynucleotides or other nucleic acids structures, may be generated using the literature methods described above. A three dimensional structure of “small” molecules or other compounds can be generated and a low energy conformation can be obtained from a short molecular dynamics minimization. These three dimensional structures can be stored in a relational database. The compounds upon which three dimensional structures are constructed can be proprietary, commercially available, or virtual.

In some embodiments of the invention, a compound data set comprising numerical representations of the three dimensional structure of a plurality of organic compounds is provided by, for example, Converter (MSI, San Diego) from two dimensional compound libraries generated by, for example, a computer program modified from a commercial program. Other suitable databases can be constructed by converting two dimensional structures of chemical compounds into three dimensional structures, as described above. The end result is the conversion of a two dimensional structure of organic compounds into numerical representations of the three dimensional structures of a plurality of organic (compounds. These numerical representations are presented as a compound data set.

After both the numerical representations of the three-dimensional structure of the polynucleotides comprising the molecular interaction sites and the compound data set comprising numerical representations of the three dimensional structures of a plurality of organic compounds are obtained, the numerical representations of the molecular interaction sites are compared with members of the compound data set to generate a hierarchy of the organic compounds. The hierarchy is ranked in accordance with the ability of the organic compounds to form physical interactions with the molecular interaction site. Preferably, the comparing is carried out seriatim upon the members of the compound data set. In accordance with some embodiments, the comparison can be performed with a plurality of polynucleotides comprising molecular interaction sites at the same time.

A variety of theoretical and computational methods are known by those skilled in the art to study and optimize the interactions of “small” molecules or organic compounds with biological targets such as nucleic acids. These structure-based drug design tools have been very useful in modelling the interactions of proteins with small molecule ligands and in optimizing these interactions. Typically this type of study has been performed when the structure of the protein receptor was known by querying individual small molecules, one at a time, against this receptor. Usually these small molecules had either been co-crystallized with the receptor, were related to other molecules that had been co-crystallized or were molecules for which some body of knowledge existed concerning their interactions with the receptor. DOCK, as described above, can be used to find and identify molecules that are expected to bind to polynucleotides comprising the molecular interaction sites and, hence, hepatitis C virus RNA of interest. DOCK 4.0 is commercially available from the Regents of the University of California. Equivalent programs are also comprehended in the present invention.

The DOCK program has been widely applied to protein targets and the identification of ligands that bind to them. Typically, new classes of molecules that bind to known targets have been identified, and later verified by in vitro experiments. The DOCK software program consists of several modules, including SPHGEN (Kuntz et al., J. Mol. Biol., 1982, 161, 269) and CHEMGRID (Meng et al., J. Comput. Chem., 1992, 13, 505, each of which is incorporated herein by reference in its entirety). SPHGEN generates clusters of overlapping spheres that describe the solvent-accessible surface of the binding pocket within the target receptor. Each cluster represents a possible binding site for small molecules. CHEMGRID precalculates and stores in a grid file the information necessary for force field scoring of the interactions between binding molecule and target hepatitis C virus RNA. The scoring function approximates molecular mechanics interaction energies and consists of van der Waals and electrostatic components. DOCK uses the selected cluster of spheres to orient ligands molecules in the targeted site on hepatitis C virus RNA. Each molecule within a previously generated three dimensional database is tested in thousands of orientations within the site, and each orientation is evaluated by the scoring function. Only that orientation with the best score for each compound so screened is stored in the output file. Finally, all compounds of the database are ranked in a hierarchy in order of their scores and a collection of the best candidates may then be screened experimentally.

Using DOCK, numerous ligands have been identified for a variety of protein targets. Recent efforts in this area have resulted in reports of the use of DOCK to identify and design small molecule ligands that exhibit binding specificity for nucleic acids such as RNA double helices. While RNA plays a significant role in many diseases such as AIDS, viral and bacterial infections, few studies have been made on small molecules capable of specific RNA binding. Compounds possessing specificity for the RNA double helix, based on the unique geometry of its deep major groove, were identified using the DOCK methodology. Chen et al., Biochemistry, 1997, 36, 11402 and Kuntz et al., Acc. Chem. Res., 1994, 27, 117. Recently, the application of DOCK to the problem of ligand recognition in DNA quadruplexes has been reported. Chen et al., Proc. Natl. Acad. Sci., 1996, 93, 2635.

Preferably, individual compounds are designated as mol files, for example, and combined into a collection of in silico representations using an appropriate chemical structure program or equivalent software. These two dimensional mol files are exported and converted into three dimensional structures using commercial software such as Converter (Molecular Simulations Inc., San Diego) or equivalent software, as described above. Atom types suitable for use with a docking program such as DOCK or QXP are assigned to all atoms in the three dimensional mol file using software such as, for example, Babel, or with other equivalent software.

A low-energy conformation of each molecule is generated with software such as Discover (MSI, San Diego). An orientation search is performed by bringing each compound of the plurality of compounds into proximity with the molecular interaction site in many orientations using DOCK or QXP. A contact score is determined for each orientation, and the optimum orientation of the compound is subsequently used. Alternatively, the conformation of the compound can be determined from a template conformation of the scaffold determined previously.

The interaction of a plurality of compounds and molecular interaction sites is examined by comparing the numerical representations of the molecular interaction sites with members of the compound data set. Preferably, a plurality of compounds such as those generated by a computer program or otherwise, is compared to the molecular interaction site and undergoes random “motions” among the dihedral bonds of the compounds. Preferably about 20,000 to 100,000 compounds are compared to at least one molecular interaction site. Typically, 20,000 compounds are compared to about five molecular interaction sites and scored. Individual conformations of the three dimensional structures are placed at the target site in many orientations. Moreover, during execution of the DOCK program, the compounds and molecular interaction sites are allowed to be “flexible” such that the optimum hydrogen bonding, electrostatic, and van der Waals contacts can be realized. The energy of the interaction is calculated and stored for 10-15 possible orientations of the compounds and molecular interaction sites. QXP methodology allows true flexibility in both the ligand and target and is presently preferred.

The relative weights of each energy contribution are updated constantly to insure that the calculated binding scores for all compounds reflect the experimental binding data. The binding energy for each orientation is scored on the basis of hydrogen bonding, van der Waals contacts, electrostatics, solvation/desolvation, and the quality of the fit. The lowest-energy van der Waals, dipolar, and hydrogen bonding interactions between the compound and the molecular interaction site are determined, and summed. In some embodiments, these parameters can be adjusted according to the results obtained empirically. The binding energies for each molecule against the target are output to a relational database. The relational database contains a hierarchy of the compounds ranked in accordance with the ability of the compounds to form physical interactions with the molecular interaction site. The higher ranked compounds are better able to form physical interactions with the molecular interaction site.

In another embodiment, the highest ranking, i.e., the best fitting compounds, are selected for synthesis. In some embodiments of the invention, those compounds which are likely to have desired binding characteristics based on binding data are selected for synthesis. Preferably the highest ranking 5% are selected for synthesis. More preferably, the highest ranking 10% are selected for syntheses. Even more preferably, the highest ranking 20% are selected for synthesis. The synthesis of the selected compounds can be automated using a parallel array synthesizer or prepared using solution-phase or other solid-phase methods and instruments. In addition, the interaction of the highly ranked compounds with the nucleic acid containing the molecular interaction site is assessed as described below.

The interaction of the highly ranked organic compounds with the polynucleotide comprising the hepatitis C virus RNA molecular interaction site can be assessed by numerous methods known to those skilled in the art. For example, the highest ranking compounds can be tested for activity in high-throughput (HTS) functional and cellular screens. HTS assays can be determined by scintillation proximity, precipitation, luminescence-based formats, filtration based assays, colorometric assays, and the like. Lead compounds can then be scaled up and tested in animal models for activity and toxicity. The assessment preferably comprises mass spectrometry of a mixture of the hepatitis C virus RNA polynucleotide and at least one of the compounds or a functional bioassay.

Certain evaluation techniques employing mass spectroscopy are disclosed in International Publication WO 99/45150, which is incorporated herein by reference in its entirety, as exemplary of certain useful and mass spectrometric techniques for use herewith. It is to be specifically understood, however, that it is not essential that these particular mass spectrometric techniques be employed in order to perform the present invention. Rather, any evaluative technique may be undertaken so long as the objectives of the present invention are maintained.

In some embodiments of the invention, the highest ranking 20% of compounds from the hierarchy generated using the DOCK program or QXP are used to generate a further data set of three dimensional representations of organic compounds comprising compounds which are chemically related to the compounds ranking high in the hierarchy. Although the best fitting compounds are likely to be in the highest ranking 1%, additional compounds, up to about 20%, are selected for a second comparison so as to provide diversity (ring size, chain length, functional groups). This process insures that small errors in the molecular interaction sites are not propagated into the compound identification process. The resulting structure/score data from the highest ranking 20%, for example, is studied mathematically (clustered) to find trends or features within the compounds which enhance binding. The compounds are clustered into different groups. Chemical synthesis and screening of the compounds, described above, allows the computed DOCK or QXP scores to be correlated with the actual binding data. After the compounds have been prepared and screened, the predicted binding energy and the observed Kd values are correlated for each compound.

The results are used to develop a predictive scoring scheme, which weighs various factors (steric, electrostatic) appropriately. The above strategy allows rapid evaluation of a number of scaffolds with varying sizes and shapes of different functional groups for the high ranked compounds. In this manner, a further data set of representations of organic compounds comprising compounds which are chemically related to the organic compounds which rank high in the hierarchy can be compared to the numerical representations of the molecular interaction site to determine a further hierarchy ranked in accordance with the ability of the organic compounds to form physical interactions with the molecular interaction site. In this manner, the further data set of representations of the three dimensional structures of compound which are related to the compounds ranked high in the hierarchy are produced and have, in effect, been optimized by correlating actual binding with virtual binding. The entire cycle can be iterated as desired until the desired number of compounds highest in the hierarchy are produced.

Compounds which have been determined to have affinity and specificity for a target biomolecule, especially a target hepatitis C virus RNA or which otherwise have been shown to be able to bind to the target hepatitis C virus RNA to effect modulation thereof, can, in accordance with some embodiments of this invention, be tagged or labelled in a detectable fashion. Such labelling may include all of the labelling forms known to persons of skill in the art such as fluorophore, radiolabel, enzymatic label and many other forms. Such labelling or tagging facilitates detection of molecular interaction sites and permits facile mapping of chromosomes and other useful processes.

Some of the preferred embodiments of the invention described above are outlined below and include, but are not limited to, the following embodiments. Thus, the following examples are meant to be exemplary of some of the invention and are not meant to be limiting. As those skilled in the art will appreciate, numerous changes and modifications may be made to the embodiments of the invention without departing from the spirit of the invention. It is intended that all such variations fall within the scope of the invention.

### EXAMPLES

### Example 1

### Selection of Hepatitis C Virus RNA

To illustrate the strategy for identifying molecular interaction sites for small molecules, the hepatitis C virus RNA was used.

### Example 2

### Molecular Interaction Sites In Hepatitis C Virus RNA

Numerous molecular interaction sites have been discovered within hepatitis C virus RNA. Site 1 comprises a region of RNA comprising a first and second polynucleotide. The first polynucleotide comprises from about seven nucleotides to about nineteen nucleotides, wherein portions of the polynucleotide form a double-stranded RNA having the following features (5′ to 3′): a first side of a stem comprising from about four nucleotides to about twelve nucleotides wherein a first side of an internal loop comprising from about two nucleotides to about five nucleotides is present in the first side of the stem and wherein a bulge comprising from about one nucleotide to about two nucleotides is present in the first side of the stem. The second polynucleotide comprises from about six nucleotides to about seventeen nucleotides, wherein portions of the polynucleotide form a double-stranded RNA having the following features (5′ to 3′): a second side of the stem comprising from about four nucleotides to about twelve nucleotides wherein a second side of the internal loop comprising from about two nucleotides to about five nucleotides is present in the second side of the stem.

In regard to site 1, the first polynucleotide preferably comprises twelve nucleotides, wherein portions of the polynucleotide form a double-stranded RNA having the following features (5′ to 3′): a first side of a stem comprising eight nucleotides wherein a first side of an internal loop comprising three nucleotides is present between the fourth and fifth nucleotides of the first side of the stem and wherein a bulge comprising one nucleotide is present between the seventh and eighth nucleotides of the first side of the stem. Preferably, the first polynucleotide comprises the sequence 5′-gaggaacuncug-3′ (SEQ ID NO:1) (bolded nucleotides indicate preferred basepairing; n is any nucleotide). The second polynucleotide preferably comprises eleven nucleotides, wherein portions of the polynucleotide form a double-stranded RNA having the following features (5′ to 3′): a second side of the stem comprising eight nucleotides wherein a second side of the internal loop comprising three nucleotides is present between the fourth and fifth nucleotides of the second side of the stem. Preferably, the second polynucleotide comprises the sequence 5′-cguncagccuc-3′ (SEQ ID NO:2) (bolded nucleotides indicate preferred basepairing; n is any nucleotide).

Site 2 comprises a region of RNA comprising a first and second polynucleotide. The first polynucleotide comprises from about five nucleotides to about fourteen nucleotides, wherein portions of the polynucleotide form a double-stranded RNA having the following features (5′ to 3′): a first side of a stem comprising from about three nucleotides to about nine nucleotides wherein a first side of an internal loop comprising from about two nucleotides to about five nucleotides is present in the first side of the stem. The second polynucleotide comprises from about five nucleotides to about fifteen nucleotides, wherein portions of the polynucleotide form a double-stranded RNA having the following features (5′ to 3′): a second side of the stem comprising from about three nucleotides to about nine nucleotides wherein a second side of the internal loop comprising from about two nucleotides to about six nucleotides is present in the second side of the stem.

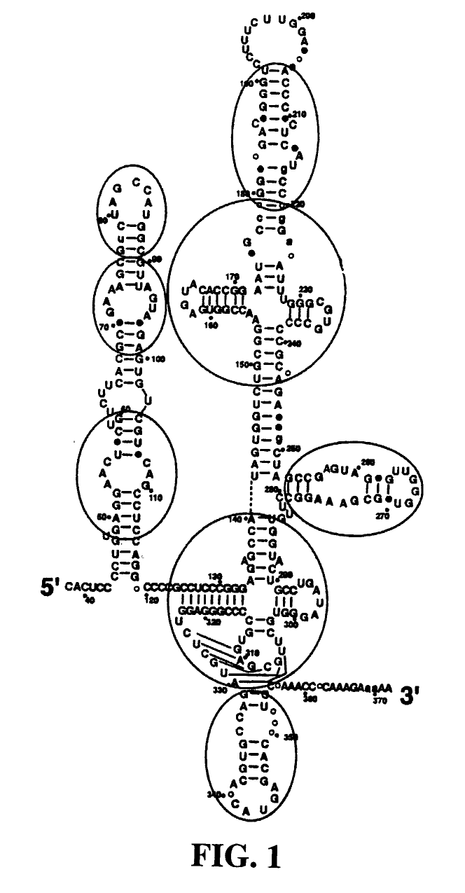

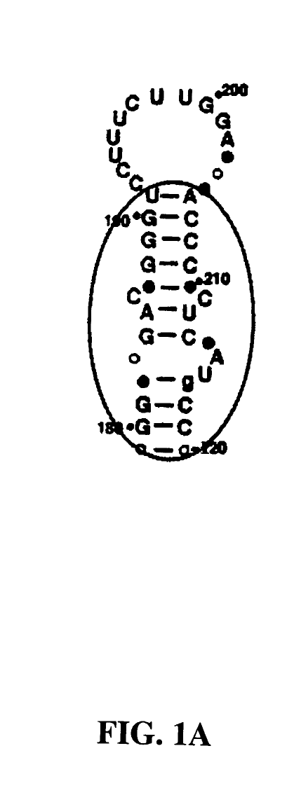

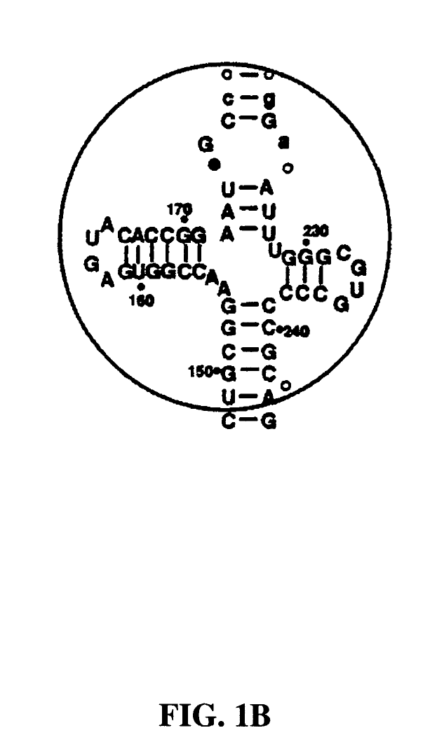

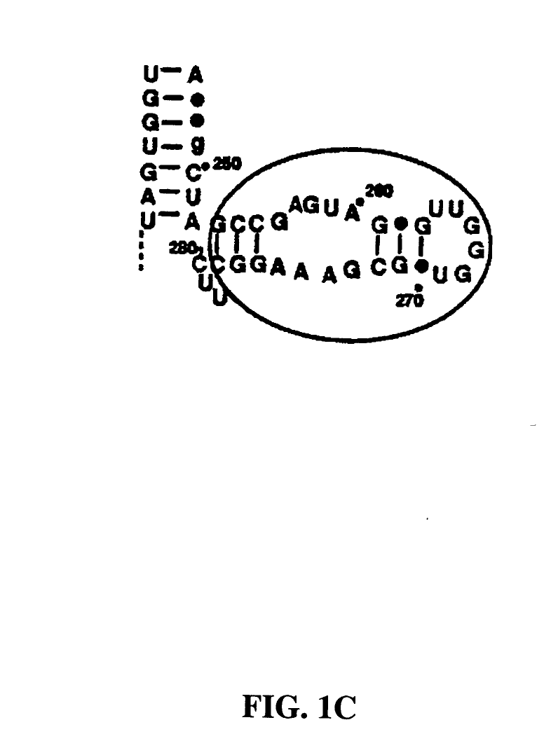

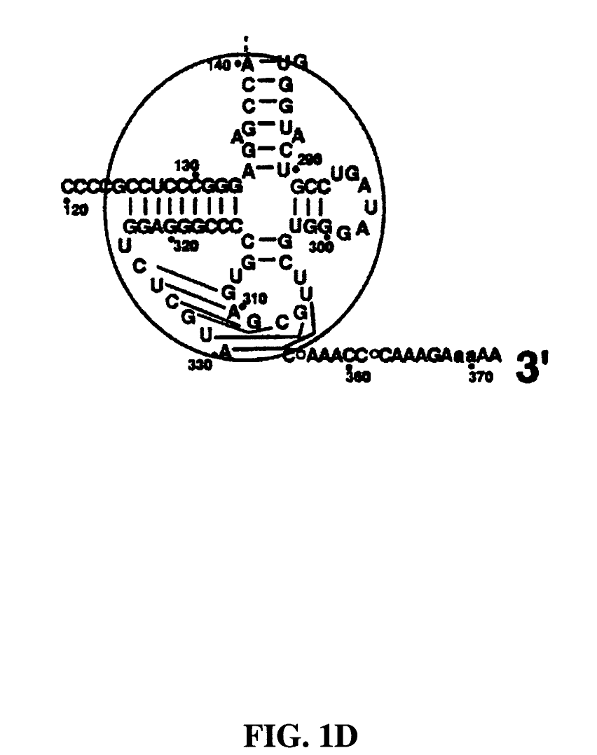

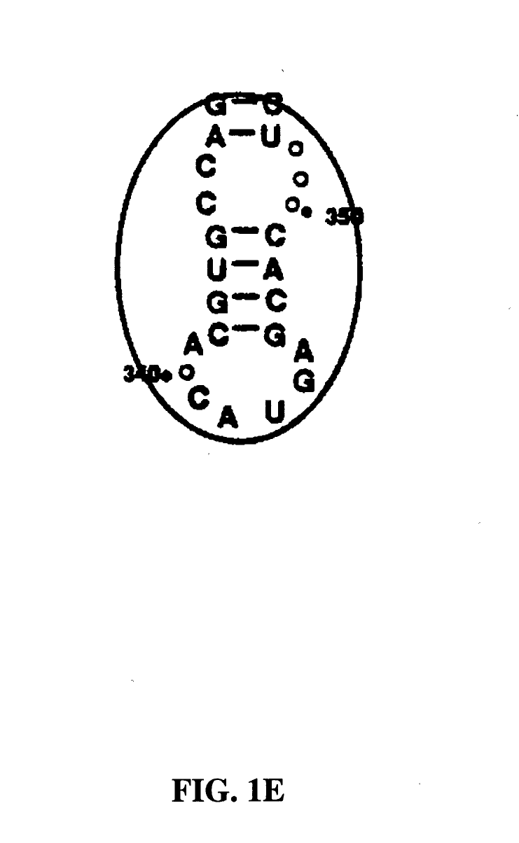

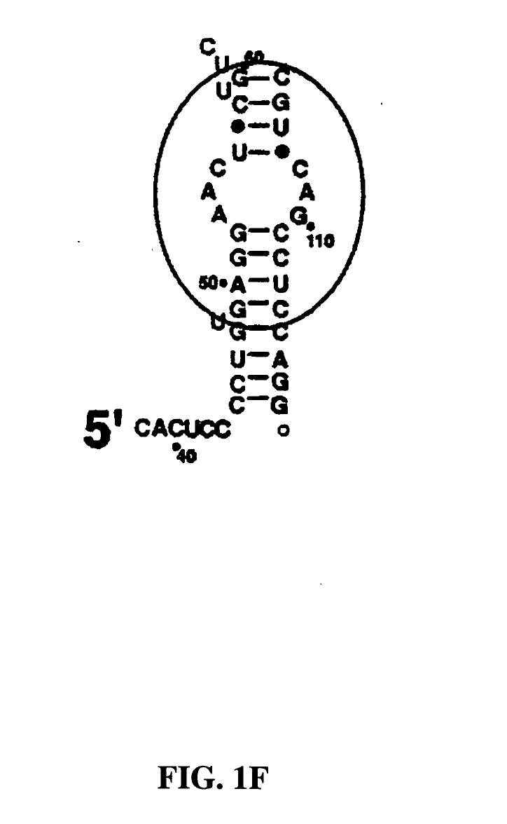

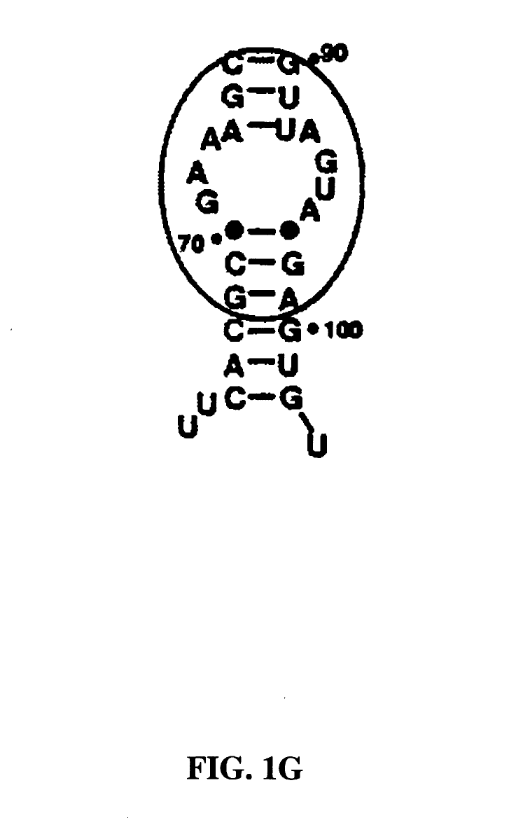

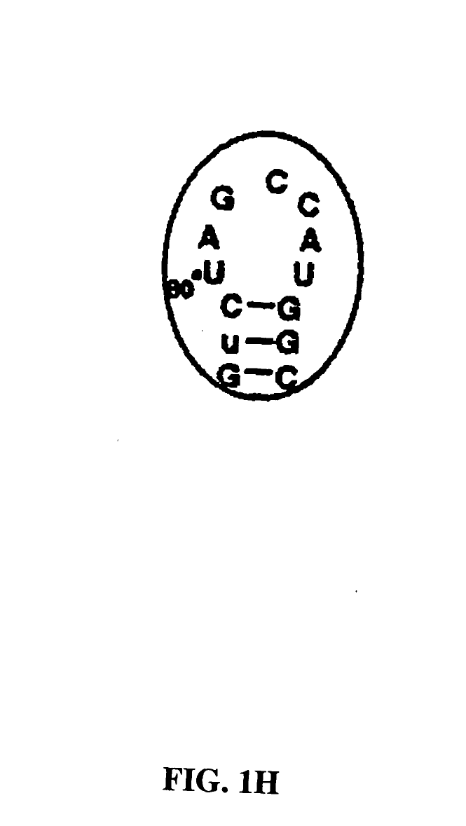

In regard to site 2, the first polynucleotide preferably comprises nine nucleotides, wherein portions of the polynucleotide form a double-stranded RNA having the following features (5′ to 3′): a first side of a stem comprising six nucleotides wherein a first side of an internal loop comprising three nucleotides is present between the third and fourth nucleotides of the first side of the stem. Preferably, the first polynucleotide comprises the sequence 5′-gcngaaagc-3′ (bolded nucleotides indicate preferred basepairing; n is any nucleotide). The second polynucleotide preferably comprises ten nucleotides, wherein portions of the polynucleotide form a double-stranded RNA having the following features (5′ to 3′): a second side of the stem comprising six nucleotides wherein a second side of the internal loop comprising four nucleotides is present between the third and fourth nucleotides of the second side of the stem. Preferably, the second polynucleotide comprises the sequence 5′-guuaguanga-3′ (SEQ ID NO:3) (bolded nucleotides indicate preferred basepairing; n is any nucleotide). Site 2 is present in HCV RNA (FIG. 1).

Site 3 comprises a region of RNA comprising a polynucleotide comprising from about eight nucleotides to about twenty nucleotides, wherein portions of the polynucleotide form a double-stranded RNA having the following features (5′ to 3′): a first side of a stem comprising from about two nucleotides to about five nucleotides, a terminal loop comprising from about four nucleotides to about ten nucleotides, and a second side of the stem comprising from about two nucleotides to about five nucleotides.

In regard to site 3, the polynucleotide preferably comprises thirteen nucleotides, wherein portions of the polynucleotide form a double-stranded RNA having the following features (5′ to 3′): a first side of a stem comprising three nucleotides, a terminal loop comprising seven nucleotides, and a second side of the stem comprising three nucleotides. Preferably, the polynucleotide comprises the sequence 5′-gucuagccauggc-3′ (SEQ ID NO:4) (bolded nucleotides indicate preferred basepairing). Site 3 is present in HCV RNA (FIG. 1).

Site 4 comprises a region of RNA comprising a first and second polynucleotide. The first polynucleotide comprises from about eight nucleotides to about twenty nucleotides, wherein portions of the polynucleotide form a double-stranded RNA having the following features (5′ to 3′): a first side of a stem comprising from about six nucleotides to about sixteen nucleotides wherein a first side of a first internal loop comprising from about one nucleotide to about two nucleotides is present in the first side of the stem and wherein a first side of a second internal loop comprising from about one nucleotide to about two nucleotides is present in the first side of the stem. The second polynucleotide comprises from about nine nucleotides to about twenty three nucleotides, wherein portions of the polynucleotide form a double-stranded RNA having the following features (5′ to 3′): a second side of the stem comprising from about six nucleotides to about sixteen nucleotides wherein a second side of the second internal loop comprising from about one nucleotide to about two nucleotides is present in the second side of the stem and wherein a second side of the first internal loop comprising from about two nucleotides to about five nucleotides is present in the second side of the stem.

In regard to site 4, the first polynucleotide preferably comprises thirteen nucleotides, wherein portions of the polynucleotide form a double-stranded RNA having the following features (5′ to 3′): a first side of a stem comprising eleven nucleotides wherein a first side of a first internal loop comprising one nucleotide is present between the fourth and fifth nucleotides of the first side of the stem and wherein a first side of a second internal loop comprising one nucleotide is present between the sixth and seventh nucleotides of the first side of the stem. Preferably, the first polynucleotide comprises the sequence 5′-nggnngacngggu-3′ (SEQ ID NO:5) (bolded nucleotides indicate preferred basepairing; n is any nucleotide). The second polynucleotide preferably comprises fifteen nucleotides, wherein portions of the polynucleotide form a double-stranded RNA having the following features (5′ to 3′): a second side of the stem comprising eleven nucleotides wherein a second side of the second internal loop comprising one nucleotide is present between the fifth and sixth nucleotides of the second side of the stem and wherein a second side of the first internal loop comprising three nucleotides is present between the seventh and eighth nucleotides of the second side of the stem. Preferably, the second polynucleotide comprises the sequence 5′-acccncucnaugccn-3′ (SEQ ID NO:6) (bolded nucleotides indicate preferred basepairing; n is any nucleotide). Site 4 is present in HCV RNA (FIG. 1).

Site 5 comprises a region of RNA comprising a first and second polynucleotide. The first polynucleotide comprises from about sixteen nucleotides to about forty six nucleotides, wherein portions of the polynucleotide form a double-stranded RNA having the following features (5′ to 3′): a first side of a first stem comprising from about three nucleotides to about seven nucleotides, a bulge comprising from about one nucleotide to about three nucleotides, a first side of a second stem comprising from about three nucleotides to about nine nucleotides, a first terminal loop comprising from about two nucleotides to about six nucleotides, a second side of the second stem comprising from about three nucleotides to about nine nucleotides, and a first side of a third stem comprising from about three nucleotides to about nine nucleotides wherein a first side of an internal loop comprising from about one nucleotide to about three nucleotides is present in the first side of the third stem. The second polynucleotide comprises from about fourteen nucleotides to about thirty seven nucleotides, wherein portions of the polynucleotide form a double-stranded RNA having the following features (5′ to 3′): a second side of the third stem comprising from about three nucleotides to about nine nucleotides wherein a second side of the internal loop comprising from about one nucleotide to about three nucleotides is present in the second side of the third stem, a bulge comprising from about one nucleotide to about two nucleotides, a first side of a fourth stem comprising from about two nucleotides to about five nucleotides, a second terminal loop comprising from about two nucleotides to about six nucleotides, a second side of the fourth stem comprising from about two nucleotides to about five nucleotides, and a second side of the first stem comprising from about three nucleotides to about seven nucleotides.

In regard to site 5, the first polynucleotide preferably comprises thirty one nucleotides, wherein portions of the polynucleotide form a double-stranded RNA having the following features (5′ to 3′): a first side of a first stem comprising five nucleotides, a bulge comprising two nucleotides, a first side of a second stem comprising six nucleotides, a first terminal loop comprising four nucleotides, a second side of the second stem comprising six nucleotides, and a first side of a third stem comprising six nucleotides wherein a first side of an internal loop comprising two nucleotides is present between the third and fourth nucleotides of the first side of the third stem. Preferably, the first polynucleotide comprises the sequence 5′-ugcggaaccggugaguacaccggaaungccn-3′ (SEQ ID NO:7) (bolded nucleotides indicate preferred basepairing; n is any nucleotide). The second polynucleotide preferably comprises twenty four nucleotides, wherein portions of the polynucleotide form a double-stranded RNA having the following features (5′ to 3′): a second side of the third stem comprising six nucleotides wherein a second side of the internal loop comprising two nucleotides is present between the third and fourth nucleotides of the second side of the third stem, a bulge comprising one nucleotide, a first side of a fourth stem comprising three nucleotides, a second terminal loop comprising four nucleotides, a second side of the fourth stem comprising three nucleotides, and a second side of the first stem comprising five nucleotides. Preferably, the second polynucleotide comprises the sequence 5′-ngganauuugggcgugcccccgca-3′ (SEQ ID NO:8) (bolded nucleotides indicate preferred basepairing; n is any nucleotide). Site 5 is present in HCV RNA (FIG. 1).

Site 6 comprises a region of RNA comprising a polynucleotide comprising from about fourteen nucleotides to about forty nucleotides, wherein portions of the polynucleotide form a double-stranded RNA having the following features (5′ to 3′): a first side of a stem comprising from about three nucleotides to about nine nucleotides wherein a first side of an internal loop comprising from about three nucleotides to about seven nucleotides is present in the first side of the stem, a terminal loop comprising from about three nucleotides to about nine nucleotides, and a second side of the stem comprising from about three nucleotides to about nine nucleotides wherein a second side of the internal loop comprising from about two nucleotides to about six nucleotides is present in the second side of the stem.

In regard to site 6, the polynucleotide preferably comprises twenty seven nucleotides, wherein portions of the polynucleotide form a double-stranded RNA having the following features (5′ to 3′): a first side of a stem comprising six nucleotides wherein a first side of an internal loop comprising five nucleotides is present between the third and fourth nucleotides of the first side of the stem, a terminal loop comprising six nucleotides, and a second side of the stem comprising six nucleotides wherein a second side of the internal loop comprising four nucleotides is present between the third and fourth nucleotides of the second side of the stem. Preferably, the polynucleotide comprises the sequence 5′-gccgaguagnguugggungcgaaaggc-3′ (SEQ ID NO:9) (bolded nucleotides indicate preferred basepairing; n is any nucleotide). Site 6 is present in HCV RNA (FIG. 1).

Site 7 comprises a region of RNA comprising a first and second polynucleotide. The first polynucleotide comprises from about ten nucleotides to about twenty six nucleotides, wherein portions of the polynucleotide form a double-stranded RNA having the following features (5′ to 3′): a dangling region comprising from about one nucleotide to about two nucleotides, a first side of a first stem comprising from about five nucleotides to about thirteen nucleotides, and a first side of a second stem comprising from about three nucleotides to about nine nucleotides wherein a first side of an internal loop comprising from about one nucleotide to about two nucleotides is in the first side of the second stem. The second polynucleotide comprises from about twenty six nucleotides to about seventy one nucleotides, wherein portions of the polynucleotide form a double-stranded RNA having the following features (5′ to 3′): a second side of the second stem comprising from about three nucleotides to about nine nucleotides wherein a second side of the internal loop comprising from about one nucleotide to about two nucleotides is present in the second side of the second stem, a first side of a third stem comprising from about two nucleotides to about five nucleotides, a first terminal loop comprising from about three nucleotides to about nine nucleotides, a second side of the third stem comprising from about two nucleotides to about five nucleotides, a first side of a fourth stem comprising from about one nucleotide to about three nucleotides, a second terminal loop comprising from about four nucleotides to about twelve nucleotides, a second side of the fourth stem comprising from about one nucleotide to about three nucleotides, a second side of the first stem comprising from about five nucleotides to about thirteen nucleotides, and a dangling region comprising from about four nucleotides to about ten nucleotides.

In regard to site 7, the first polynucleotide preferably comprises seventeen nucleotides, wherein portions of the polynucleotide form a double-stranded RNA having the following features (5′ to 3′): a dangling region comprising one nucleotide, a first side of a first stem comprising nine nucleotides, and a first side of a second stem comprising six nucleotides wherein a first side of an internal loop comprising one nucleotide is present between the second and third nucleotides of the first side of the second stem. Preferably, the first polynucleotide comprises the sequence 5′-gccucccgggagagcca-3′ (SEQ ID NO:10) (bolded nucleotides indicate preferred basepairing). The second polynucleotide preferably comprises forty seven nucleotides, wherein portions of the polynucleotide form a double-stranded RNA having the following features (5′ to 3′): a second side of the second stem comprising six nucleotides wherein a second side of the internal loop comprising one nucleotide is present between the fourth and fifth nucleotides of the second side of the second stem, a first side of a third stem comprising three nucleotides, a first terminal loop comprising six nucleotides, a second side of the third stem comprising three nucleotides, a first side of a fourth stem comprising two nucleotides, a second terminal loop comprising eight nucleotides, a second side of the fourth stem comprising two nucleotides, a second side of the first stem comprising nine nucleotides, and a dangling region comprising seven nucleotides. Preferably, the second polynucleotide comprises the sequence 5′-ugguacugccugauagggugcuugcgagugccccgggaggucucgua-3′ (SEQ ID NO:11) (bolded nucleotides indicate preferred basepairing). Site 7 is present in HCV RNA (FIG. 1).

Site 8 comprises a region of RNA comprising a polynucleotide comprising from about thirteen nucleotides to about thirty six nucleotides, wherein portions of the polynucleotide form a double-stranded RNA having the following features (5′ to 3′): a first side of a stem comprising from about three nucleotides to about nine nucleotides wherein a first side of an internal loop comprising from about one nucleotide to about three nucleotides is present in the first side of the stem, a terminal loop comprising from about four nucleotides to about ten nucleotides, and a second side of the stem comprising from about three nucleotides to about nine nucleotides wherein a second side of the internal loop comprising from about two nucleotides to about five nucleotides is present in the second side of the stem.

In regard to site 8, the polynucleotide preferably comprises twenty four nucleotides, wherein portions of the polynucleotide form a double-stranded RNA having the following features (5′ to 3′): a first side of a stem comprising six nucleotides wherein a first side of an internal loop comprising two nucleotides is present between the second and third nucleotides of the first side of the stem, a terminal loop comprising seven nucleotides, and a second side of the stem comprising six nucleotides wherein a second side of the internal loop comprising three nucleotides is present between the fourth and fifth nucleotides of the second side of the stem. Preferably, the polynucleotide comprises the sequence 5′-gaccgugcancaugagcacnnnuc-3′ (SEQ ID NO:12) (bolded nucleotides indicate preferred basepairing; n is any nucleotide). Site 8 is present in HCV RNA (FIG. 1).

What is claimed is:

1. A composition comprising a first polynucleotide and a second polynucleotide wherein:
the first polynucleotide comprises from about seven nucleotides to about sixty nine nucleotides and comprises a secondary structure defined by: a first side of a stem comprising from about four nucleotides to about twelve nucleotides wherein a first side of an internal loop comprising from about two nucleotides to about five nucleotides is present in the first side of the stem and wherein a bulge comprising from about one nucleotide to about two nucleotides is present in the first side of the stem; and
the second polynucleotide comprises from about six nucleotides to about sixty seven nucleotides and comprises a secondary structure defined by: a second side of the stem comprising from about four nucleotides to about twelve nucleotides wherein a second side of the internal loop comprising from about two nucleotides to about five nucleotides is present in the second side of the stem.

2. The composition of claim 1 wherein the first polynucleotide comprises at least twelve nucleotides but not more than sixty two nucleotides and comprises a secondary structure defined by: a first side of a stem comprising eight nucleotides wherein a first side of an internal loop comprising three nucleotides is present between the fourth and fifth nucleotides of the first side of the stem and wherein a bulge comprising one nucleotide is present between the seventh and eighth nucleotides of the first side of the stem; and
wherein the second polynucleotide comprises at least eleven nucleotides but not more than sixty one nucleotides and comprises a secondary structure defined by: a second side of the stem comprising eight nucleotides wherein a second side of the internal loop comprising three nucleotides is present between the fourth and fifth nucleotides of the second side of the stem.

3. The composition of claim 2 wherein the first polynucleotide comprises SEQ ID NO:1.

4. The composition of claim 2 wherein the second polynucleotide comprises SEQ ID NO:2.

5. A composition comprising a first polynucleotide and a second polynucleotide wherein:
the first polynucleotide comprises from about five nucleotides to about sixty four nucleotides and comprises a secondary structure defined by: a first side of a stem comprising from about three nucleotides to about nine nucleotides wherein a first side of an internal loop comprising from about two nucleotides to about five nucleotides is present in the first side of the stem; and
the second polynucleotide comprises from about five nucleotides to about sixty five nucleotides and comprises a secondary structure defined by: a second side of the stem comprising from about three nucleotides to about nine nucleotides wherein a second side of the internal loop comprising from about two nucleotides to about six nucleotides is present in the second side of the stem.

6. The composition of claim 5 wherein the first polynucleotide comprises at least nine nucleotides but not more than fifty nine nucleotides and comprises a secondary structure defined by: a first side of a stem comprising six nucleotides wherein a first side of an internal loop comprising three nucleotides is present between the third and fourth nucleotides of the first side of the stem; and
wherein the second polynucleotide comprises at least ten nucleotides but not more than sixty nucleotides and comprises a secondary structure defined by: a second side of the stem comprising six nucleotides wherein a second side of the internal loop comprising four nucleotides is present between the third and fourth nucleotides of the second side of the stem.

7. The composition of claim 6 wherein the first polynucleotide comprises 5′-gcngaaagc-3′.

8. The composition of claim 6 wherein the second polynucleotide comprises SEQ ID NO:3.

9. A polynucleotide comprising from about eight nucleotides to about seventy nucleotides comprising a secondary structure defined by: a first side of a stem comprising from about two nucleotides to about five nucleotides, a terminal loop comprising from about four nucleotides to about ten nucleotides, and a second side of the stem comprising from about two nucleotides to about five nucleotides.

10. The polynucleotide of claim 9 comprising at least thirteen nucleotides and up to sixty three nucleotides comprising a secondary structure defined by: a first side of a stem comprising three nucleotides, a terminal loop comprising seven nucleotides, and a second side of the stem comprising three nucleotides.

11. The polynucleotide of claim 10 comprising SEQ ID NO:4.

12. A composition comprising a first polynucleotide and a second polynucleotide wherein:
the first polynucleotide comprises from about eight nucleotides to about seventy nucleotides and comprises a secondary structure defined by: a first side of a stem comprising from about six nucleotides to about sixteen nucleotides wherein a first side of a first internal loop comprising from about one nucleotide to about two nucleotides is present in the first side of the stem and wherein a first side of a second internal loop comprising from about one nucleotide to about two nucleotides is present in the first side of the stem; and
the second polynucleotide comprises from about nine nucleotides to about seventy three nucleotides and comprises a secondary structure defined by: a second side of the stem comprising from about six nucleotides to about sixteen nucleotides wherein a second side of the second internal loop comprising from about one nucleotide to about two nucleotides is present in the second side of the stem and wherein a second side of the first internal loop comprising from about two nucleotides to about five nucleotides is present in the second side of the stem.

13. The composition of claim 12 wherein the first polynucleotide comprises at least thirteen nucleotides but not more than sixty three nucleotides and comprises a secondary structure defined by: a first side of a stem comprising eleven nucleotides wherein a first side of a first internal loop comprising one nucleotide is present between the fourth and fifth nucleotides of the first side of the stem and wherein a first side of a second internal loop comprising one nucleotide is present between the sixth and seventh nucleotides of the first side of the stem; and
wherein the second polynucleotide comprises at least fifteen nucleotides but not more than sixty five nucleotides and comprises a secondary structure defined by: a second side of the stem comprising eleven nucleotides wherein a second side of the second internal loop comprising one nucleotide is present between the fifth and sixth nucleotides of the second side of the stem and wherein a second side of the first internal loop comprising three nucleotides is present between the seventh and eighth nucleotides of the second side of the stem.

14. The composition of claim 13 wherein the first polynucleotide comprises SEQ ID NO:5.

15. The composition of claim 13 wherein the second polynucleotide comprises SEQ ID NO:6.

16. A composition comprising a first polynucleotide and a second polynucleotide wherein:
the first polynucleotide comprises from about sixteen nucleotides to about ninety six nucleotides and comprises a secondary structure defined by: a first side of a first stem comprising from about three nucleotides to about seven nucleotides, a bulge comprising from about one nucleotide to about three nucleotides, a first side of a second stem comprising from about three nucleotides to about nine nucleotides, a first terminal loop comprising from about two nucleotides to about six nucleotides, a second side of the second stem comprising from about three nucleotides to about nine nucleotides, and a first side of a third stem comprising from about three nucleotides to about nine nucleotides wherein a first side of an internal loop comprising from about one nucleotide to about three nucleotides is present between the third and fourth nucleotides of the first side of the third stem; and
the second polynucleotide comprises from about fourteen nucleotides to about eighty seven nucleotides and comprises a secondary structure defined by: a second side of the third stem comprising from about three nucleotides to about nine nucleotides wherein a second side of the internal loop comprising from about one nucleotide to about three nucleotides is present between the third and fourth nucleotides of the second side of the third stem, a bulge comprising from about one nucleotide to about two nucleotides, a first side of a fourth stem comprising from about two nucleotides to about five nucleotides, a second terminal loop comprising from about two nucleotides to about six nucleotides, a second side of the fourth stem comprising from about two nucleotides to about five nucleotides, and a second side of the first stem comprising from about three nucleotides to about seven nucleotides.

17. The composition of claim 16 wherein the first polynucleotide comprises at least thirty one nucleotides but not more than eighty one nucleotides and comprises a secondary structure defined by: a first side of a first stem comprising five nucleotides, a bulge comprising two nucleotides, a first side of a second stem comprising six nucleotides, a first terminal loop comprising four nucleotides, a second side of the second stem comprising six nucleotides, and a first side of a third stem comprising six nucleotides wherein a first side of an internal loop comprising two nucleotides is present between the third and fourth nucleotides of the first side of the third stem; and
wherein the second polynucleotide comprises at least twenty four nucleotides but not more than seventy four nucleotides and comprises a secondary structure defined by: a second side of the third stem comprising six nucleotides wherein a second side of the internal loop comprising two nucleotides is present between the third and fourth nucleotides of the second side of the third stem, a bulge comprising one nucleotide, a first side of a fourth stem comprising three nucleotides, a second terminal loop comprising four nucleotides, a second side of the fourth stem comprising three nucleotides, and a second side of the first stem comprising five nucleotides.

18. The composition of claim 17 wherein the first polynucleotide comprises SEQ ID NO:7.

19. The composition of claim 17 wherein the second polynucleotide comprises SEQ ID NO:8.

20. A polynucleotide comprising from about fourteen nucleotides to about ninety nucleotides comprising a secondary structure defined by: a first side of a stem comprising from about three nucleotides to about nine nucleotides wherein a first side of an internal loop comprising from about three nucleotides to about seven nucleotides is present in the first side of the stem, a terminal loop comprising from about three nucleotides to about nine nucleotides, and a second side of the stem comprising from about three nucleotides to about nine nucleotides wherein a second side of the internal loop comprising from about two nucleotides to about six nucleotides is present in the second side of the stem.

21. The polynucleotide of claim 20 comprising at least twenty seven nucleotides and up to seventy seven nucleotides comprising a secondary structure defined by: a first side of a stem comprising six nucleotides wherein a first side of an internal loop comprising five nucleotides is present between the third and fourth nucleotides of the first side of the stem, a terminal loop comprising six nucleotides, and a second side of the stem comprising six nucleotides wherein a second side of the internal loop comprising four nucleotides is present between the third and fourth nucleotides of the second side of the stem.

22. The polynucleotide of claim 21 comprising SEQ ID NO:9.

23. A composition comprising a first polynucleotide and a second polynucleotide wherein:
the first polynucleotide comprises from about ten nucleotides to about seventy six nucleotides and comprises a secondary structure defined by: a dangling region comprising from about one nucleotide to about two nucleotides, a first side of a first stem comprising from about five nucleotides to about thirteen nucleotides, and a first side of a second stem comprising from about three nucleotides to about nine nucleotides wherein a first side of an internal loop comprising from about one nucleotide to about two nucleotides is present in the first side of the second stem; and
the second polynucleotide comprises from about twenty six nucleotides to about one hundred twenty one nucleotides and comprises a secondary structure defined by: a second side of the second stem comprising from about three nucleotides to about nine nucleotides wherein a second side of the internal loop comprising from about one nucleotide to about two nucleotides is present in the second side of the second stem, a first side of a third stem comprising from about two nucleotides to about five nucleotides, a first terminal loop comprising from about three nucleotides to about nine nucleotides, a second side of the third stem comprising from about two nucleotides to about five nucleotides, a first side of a fourth stem comprising from about one nucleotide to about three nucleotides, a second terminal loop comprising from about four nucleotides to about twelve nucleotides, a second side of the fourth stem comprising from about one nucleotide to about three nucleotides, a second side of the first stem comprising from about five nucleotides to about thirteen nucleotides, and a dangling region comprising from about four nucleotides to about ten nucleotides.

24. The composition of claim 23 wherein the first polynucleotide comprises at least seventeen nucleotides but not more than sixty seven nucleotides and comprises a secondary structure defined by: a dangling region comprising one nucleotide, a first side of a first stem comprising nine nucleotides, and a first side of a second stem comprising six nucleotides wherein a first side of an internal loop comprising one nucleotide is present between the second and third nucleotides of the first side of the second stem; and
wherein the second polynucleotide comprises at least forty seven nucleotides but not more than ninety seven nucleotides and comprises a secondary structure defined by: a second side of the second stem comprising six nucleotides wherein a second side of the internal loop comprising one nucleotide is present between the fourth and fifth nucleotides of the second side of the second stem, a first side of a third stem comprising three nucleotides, a first terminal loop comprising six nucleotides, a second side of the third stem comprising three nucleotides, a first side of a fourth stem comprising two nucleotides, a second terminal loop comprising eight nucleotides, a second side of the fourth stem comprising two nucleotides, a second side of the first stem comprising nine nucleotides, and a dangling region comprising seven nucleotides.

25. The composition of claim 24 wherein the first polynucleotide comprises SEQ ID NO:10.

26. The composition of claim 24 wherein the second polynucleotide comprises SEQ ID NO:11.

27. A polynucleotide comprising from about thirteen nucleotides to about eighty six nucleotides comprising a secondary structure defined by: a first side of a stem comprising from about three nucleotides to about nine nucleotides wherein a first side of an internal loop comprising from about one nucleotide to about three nucleotides is present in the first side of the stem, a terminal loop comprising from about four nucleotides to about ten nucleotides, and a second side of the stem comprising from about three nucleotides to about nine nucleotides wherein a second side of the internal loop comprising from about two nucleotides to about five nucleotides is present in the second side of the stem.

28. The polynucleotide of claim 27 comprising at least twenty four nucleotides and up to one hundred twenty four nucleotides comprising a secondary structure defined by: a first side of a stem comprising six nucleotides wherein a first side of an internal loop comprising two nucleotides is present between the second and third nucleotides of the first side of the stem, a terminal loop comprising seven nucleotides, and a second side of the stem comprising six nucleotides wherein a second side of the internal loop comprising three nucleotides is present between the fourth and fifth nucleotides of the second side of the stem.

29. The polynucleotide of claim 28 comprising SEQ ID NO:12.
